(19) United States
(12) Patent Application Publication
(10) Pub. No.: US 2006/0173405 A1
(43) Pub. Date: Aug. 3, 2006

(54) Single use lubricant dispenser

(76) Inventor: Lisa Haithcock, Chesterfield, MO (US)

Correspondence Address: BUHLER ASSOCIATES;BUHLER, KIRK A., 1101 CALIFORNIA AVE., SUITE 208, CORONA, CA, 92881, US

(21) Appl. No.: 11/009,341

(22) Filed: Dec. 10, 2004

(51) Int. Cl. A61M 5/178

(52) U.S. Cl. 604/37

(57) ABSTRACT

A single use lubricant and applicator is disclosed where the lubricant and applicator consist of a single package. The package is a pre-measure quantity of sterile lubricant in a closed container. The user opens the container, uncoils the applicator, and then squeezes the lubricant reservoir attached to the applicator to dispense the lubricant. The package can be applied with one hand. The single use package allows minimal storage space and less trash. The combined reservoir and applicator requires no assembly and potential contamination. The invention is also suitable for use with flavored and un-flavored lubricants, creams, antibiotics, cosmetics and ointment.

### FIELD OF THE INVENTION

This invention relates to new and useful lubricant dispenser. More particularly, the present invention relates to single use sterilized lubricant and dispenser. The invention consists of a reservoir of lubricant that is contained in a dispenser and applicator where a medical person or user can remove one package, open the package, squeeze the dispenser, and the lubricant in the dispenser would flow through and out of an applicator.

### BACKGROUND OF THE INVENTION

The prior art discloses many examples of products that include lubricants and or applicators, but none providing the combination of features disclosed and claimed herein.

Wells et al. U.S. Pat. No. 6,619,837 discloses a probe cover with a lubrication well. This patent is for probe that is used with an electric thermometer. The electric thermometer is placed into the probe cover. As the electronic thermometer is inserted into the probe cover, an internal cover located inside the outer cover grips the electronic thermometer and is removed with the electronic thermometer. The internal cover includes a lubricant that makes insertion of the electronic thermometer easier. This invention provides a single use lubricant, but it is only intended for use with probes, and cannot be used for manual examinations, and requires an inner and outer cover.

House U.S. Pat. No. 6,090,075 discloses a disposable urinary catheterization assembly. This patent includes a lubricant located on or around the urinary catheter. The urinary catheter is intended for a single use, and as the catheter assembly is opened, lubricant is deposited onto the catheter guide, so insertion of the catheter is easier. This invention provides a single use lubricant, but it is only intended for use with the catheter, and cannot be used for manual examinations, and requires the catheter bag to retain the lubricant.

Hussey U.S. Pat. No. 4,75,288 discloses a disposable enema unit. This patent includes a lubricant located on or around the tip of the enema unit. The enema unit is disposable and intended for a single use. As the enema unit is prepared for use, lubricant is deposited onto the rectal tip, so insertion of the rectal tip is easier. This invention provides a single use lubricant, but it is only intended for use with the enema unit, and cannot be used for manual examinations, and requires the disposable enema unit to retain the lubricant.

Uson U.S. Pat. No. 4,269,310 discloses a septic container and manipulator for a urethral catheter having an integral antiseptic solution and lubricant. This patent includes a lubricant located on or around the urethral catheter. The urethral catheter is intended for a single use, and as the catheter assembly is opened, lubricant is deposited onto the catheter guide, so insertion of the catheter is easier. This invention provides a single use lubricant, but it is only intended for use with the catheter, and cannot be used for manual examinations, and requires the urethral catheter and manipulator to retain the lubricant.

Lubricants are available today that are sold in tubes. Some products such as KY Jelly, petroleum jelly, Preparation H are sold with a separate nozzle that can be inserted into the rectal cavity to dispense the product. These products don't include a built-in nozzle for lubricant placement. While these products can be used once and discarded, they are not intended for use by medical professionals where only a small amount of lubricant is needed, and a sterile environment is required.

### BRIEF SUMMARY OF THE INVENTION

One of the objects of this invention is to provide a single use lubricant and applicator that can be used and disposed easily.

Another object of the invention is to provide a single use lubricant that includes an integrated applicator that allows the lubricant to be easily placed without requiring assembly of the applicator onto the lubricant reservoir

Another object of the invention is to provide a lubricant that eliminates the potential cross contamination or the transfer of diseases such as HIV or other transmitted diseased caused by re-using medical products.

Another object of the invention is to provide a single use lubricant where the lubricant can be applied using one hand by squeezing the lubricant reservoir.

Another object of the invention is to provide a lubricant where only a measured small amount of lubricant is dispensed to reduce wasted lubricant and landfill.

Another object of the invention is to provide a lubricant that is contained in a sterile package that can be opened and used without contamination of the lubricant.

Another object of the invention is to provide an application package that provides a pre-measure single use amount of lubricant, cream, anti-biotic or ointment.

Another object of the invention is to provide a lubricant and applicator where the applicator provides lubricant at the tip of the applicator and or provides lubricant to be available at the sides of the applicator.

Another object of the invention is to provide a lubricant and applicator in a coiled configuration to minimize storage volume and the applicator can be un-coiled for use.

Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is an isometric view of the single use dispenser as packaged for sale or use.

FIG. 2 is an isometric view of the single use lubricant dispenser shown expanded.

FIG. 3 is a cross sectional view of the single use lubricant dispenser.

FIG. 4 shows the single use lubricant dispenser being used.

### DETAILED DESCRIPTION OF THE INVENTION

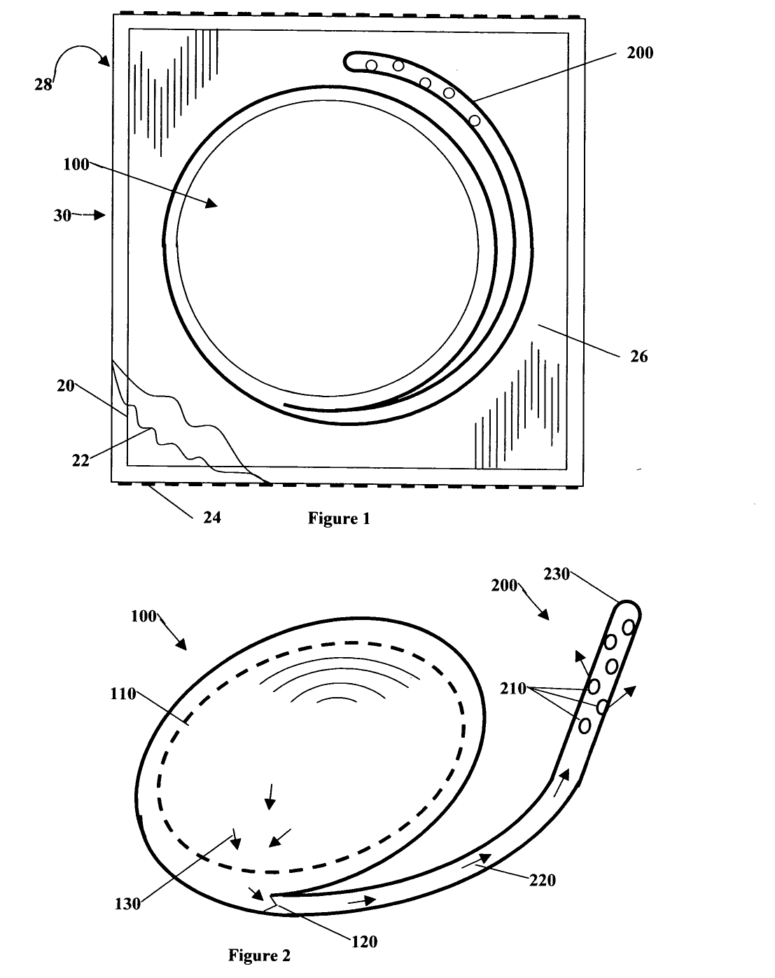

Referring to FIG. 1, that shows an isometric view of the single use dispenser as packaged for sale or use. From this figure, the single use lubricant 100 is shown inside a closed package 30. The packaging consists of a semi-rigid structure that prevents accidental crushing of the single use lubricant product. In the preferred embodiment, the packaging is made from a vacuum formed plastic sheet. The vacuum formed sheet consists of a recess 26 where the product is placed to protect it from damage. The single use lubricant dispenser is placed inside the formed plastic package 20, and a protective film 22 is placed over the single use lubricant dispenser and sealed. The sealing process retains the single use lubricant dispenser inside the package and provides a sterilized environment for the lubricant dispenser. The lubricant consists of a reservoir 100 of lubricant attached to a dispensing nozzle 200. The various components of the invention are best described by referring to FIG. 2. The packaging can consist of as individual packaged units or may be made with a tear away or perforated carrier where the perforations 24 exist between adjacent single use packages. The package may contain instructions placed on the cover or backside of the package 28. The instructions may include information on how to open the packaging, how to use and where to place the lubricant. The instructions may include medical information on the type of lubricant, the chemical mixture and the any emergency information if the lubricant is miss-used. When the single use lubricant is removed from the packaging shown in FIG. 1, the application hose is at least partially wrapped around the bulb of lubricant. The application hose can be straightened or re-shaped for use.

FIG. 2 is an isometric view of the single use lubricant shown expanded. From this figure, the lubricant reservoir 110 is shown as a bulb. The bulb 110 contains a measure amount of lubricant or ointment. The ointment is retained in the bulb portion of the invention to keep it protected from contamination. A seal 120 may exist at one end of the bulb such that when the bulb is compressed or squeezed, the material retained within the bulb opens the seal and the material within the bulb can move past the seal and into the dispenser portion of the invention. Various types of seals can be used; in addition the product may be fabricated without a seal. The seal may be one or more slits in the reservoir of the product or may be held closed by natural forces, or by a mechanical clip placed on the end of the reservoir portion of the invention. Different types of seals can be used that require varying amounts of force or pressure to release or begin to make the fluid flow from the reservoir to the dispensing tube. Once the lubricant 130 enters the dispensing portion 200 or tube 220 of the product, it continues down the tube and can exit one or more dispensing holes or slits 210. The preferred embodiment includes holes or slits, but they are not required in the invention. If the invention does not include opening, hole or slit in the side of the dispensing tube, the dispensing tube will include at least one hole in the end of the dispensing tube 230 where the fluid can be dispensed. The length of the dispensing tube can vary from as short as several millimeters to 10 or more centimeters in length. The length can be any length that allows placement of the lubricant where it will be most effective. In the preferred embodiment the bulb is shaped like an ellipse, but can be shaped like a light bulb, spherical, cylindrical, square, rectangular, or any shape that can retain fluid and allows the product to most easily be used.

The dispensing tube has a sturdy yet flexible rubber or plastic hose. The dispensing tube can be manufactured with one or more metallic spines that allow the tube to be straightened or shaped to allow for easier placement.

The fluid within the reservoir has been described as a lubricant, but the fluid can be a variety of types where a single use may be required. The fluid within the reservoir can be water soluble like KY jelly or non water soluble such as petroleum jelly. The fluid may be a cosmetic cream such as hand cream or vitamin E. The fluid may have medical purposes such as Vicks, preparation H, isopropyl alcohol or ointment. Flavoring, colors and or thermal properties can be added to the lubricant to make the product more desirable or to sell the product to enhance sexual activity. The thickness or viscosity of the fluid can vary from the consistency of water to the consistency of butter or thicker. In the preferred embodiment the volume of the lubricant is about 1.5 cc, but the lubricant can be greater or less than 1.5 cc based upon an expected single use. In the case of dispensing lubricant such as KY Jelly that is used for a medical examination such as a vaginal or proctologic exam, the lubricant can be applied to a finger or examination tool. The lubricant can be partially squeezed out of the dispensing tube, and the dispensing tube can be inserted within the vaginal or rectal cavity, and the remainder of the lubricant can be expelled from the reservoir. After sufficient amount of lubricant has been expelled from the reservoir, the dispenser is disposed to prevent cross contamination with another person. The reservoir bulb can be manufactured in a colored or clear material. It is contemplated that different colors can be used to designate different lubricants or products that are within the reservoir.

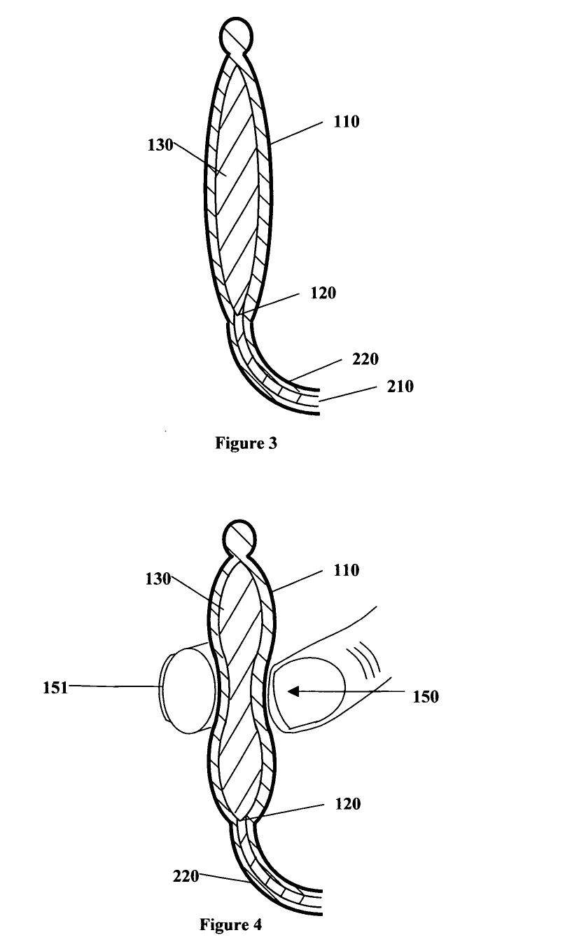

FIG. 3 is a cross sectional view of the single use lubricant dispenser. In this figure the bulb 110 is shown with the reservoir of fluid 130. The end of the reservoir has a seal 120 at one end of the bulb. The seal is the end of the bulb that prevents contamination from entering into the reservoir. As previously discussed, the seal can be a variety of configurations or methods that reduces or prevent contamination and reduces the possibility of accidentally expelling fluid from the reservoir before the dispensing tube is in place. The dispensing tube 220 is shown with a hole 210 where the lubricant is expelled.

FIG. 4 shows the single use lubricant dispenser being used where the bulb 110 containing lubricant 130. The bulb is shown being squeezed with two fingers that push in 150, 151 on the sides of the tube. The squeezing of the sides forces the lubricant out the seal 120, and into the dispensing tube 220.

Thus, specific embodiments and applications for a medical single use lubricant dispenser. It should be apparent, however, to those skilled in the art that many more modifications besides those described are possible without departing from the inventive concepts herein. The inventive subject matter, therefore, is not to be restricted except in the spirit of the appended claims.

What is claimed is:

1. A medical single use dispenser comprising:
a single use reservoir,
a yieldable seal located at one end of the reservoir,
an application mechanism located at one end of the reservoir with the yieldable seal, such that
when sufficient force is placed upon the reservoir, the seal will open, and a fluid within the reservoir will enter the application mechanism.

2. The single use reservoir from claim 1 wherein the reservoir contains more than 1 cc of fluid.

3. The single use reservoir from claim 1 wherein the reservoir contains less than 2 cc of fluid.

4. The yieldable seal located at one end of the reservoir from claim 1 wherein the seal comprises at least one yieldable slit.

5. The yieldable seal located at one end of the reservoir from claim 1 wherein the seal comprises a mechanical clip located outside the reservoir and the application mechanism.

6. The sufficient force from claim 1 wherein the force is greater than 30 grams of force.

7. The sufficient force from claim 1 wherein the force is less than greater than 100 grams of force.

8. The application mechanism from claim 1 wherein the application

9. The fluid from claim 1 wherein the fluid has medical uses.

10. The fluid from claim 1 wherein the fluid has lubricating uses.

11. The fluid from claim 1 wherein the fluid has sexual uses.

12. The fluid from claim 1 wherein the fluid has cosmetic uses.

13. The medical single use dispenser from claim 1 further includes packaging that allows dispensing of a single sealed sterile dispenser.

14. The packaging from claim 13 wherein multiple packages are perforated from each other on at least one side.

15. The medical single use dispenser from claim 1 further includes instruction on the use of the dispenser.

16. The application mechanism from claim 1 wherein the application mechanism comprises a tube.

17. The tube from claim 16 wherein the tube is longer than 1 centemeter.

18. The tube from claim 16 wherein the tube is shorter than 10 centimeters.

19. The application mechanism from claim 1 wherein the application mechanism comprises a tube with at least one opening at the side of the tube where the fluid is dispensed.

20. The application mechanism from claim 1 wherein the application mechanism comprises a tube with at least one opening at the end of the tube where the fluid is dispensed.
